(19) United States
(12) Patent Application Publication
(10) Pub. No.: US 2024/0167074 A1
(43) Pub. Date: May 23, 2024

(54) BIOCHEMICAL SELECTIVITY PROFILING AGAINST RNA HELICASES

(71) Applicant: Baylor College of Medicine, Houston, TX (US)

(72) Inventors: Thomas F. Westbrook, Houston, TX (US); Kristen Karlin, Houston, TX (US); Calla Olson, Houston, TX (US)

(21) Appl. No.: 18/549,554

(22) PCT Filed: Mar. 8, 2022
(86) PCT No.: PCT/US22/71038

§ 371 Date: Sep. 7, 2023

Related U.S. Application Data
(60) Provisional application No. 63/158,719, filed on Mar. 9, 2021.

(51) Int. Cl. C12Q 1/34; G01N 33/50

(52) U.S. Cl. CPC C12Q 1/34; G01N 33/5008; G01N 2500/04; G01N 2500/10

(57) ABSTRACT

Embodiments of the disclosure concern identification of suitable inhibitors of components of RNA metabolism, including those involved with splicing, such as RNA helicases. The disclosure includes in vitro screens for small molecule inhibitors that are selective for a group of RNA helicases but that are counter-selective for another group of RNA helicases. In specific embodiments, the disclosure includes a system for inhibitor analysis that includes assay development and optimization that leads to feasible screening of the intended target(s) and continued counter-selection against undesirable targets throughout the process.

This application claims priority to U.S. Provisional patent application Ser. No. 63/158,719, filed Mar. 9, 2021, which is incorporated by reference herein in its entirety.

### TECHNICAL FIELD

Embodiments of the disclosure include at least the fields of cell biology, molecular biology, RNA metabolism, drug design and development, drug screening, and medicine.

### BACKGROUND

RNA helicases are ATPases that participate in most aspects of RNA metabolism and processing. Recent functional genomic studies (RNAi and CRISPR) have revealed that cancer cells are uniquely dependent on RNA helicases for survival. Notably, dependency on a specific RNA helicase often varies across tumor type and genetic background, suggesting selective dependency across tissue-of-origin and/or associated oncogenic drivers. However, the mechanisms driving these tissue- and/or genotype-specific dependencies remain elusive in part because of a lack of selective and potent RNA helicase inhibitors. While RNA helicases are attractive oncology targets, limited progress has been made toward the identification of clinical-grade inhibitors, because the assays and tools required to assess compound selectivity do not yet exist.

The present disclosure addresses the need in the art by providing methods for identifying selective inhibitors for RNA helicases and other RNA metabolism components, including for medicine.

### BRIEF SUMMARY

Embodiments of the disclosure include methods and compositions that allow for drug discovery programs related to RNA metabolism, including at least being related to synthesis, folding/unfolding, modification, processing and degradation of RNA. In specific embodiments, the methods and compositions allow for drug discovery programs related to RNA processing including any modification made to RNA between its transcription and its final cellular function, such as at least RNA splicing. In some embodiments the methods and compositions concern RNA helicases as the subject of characterization and selectivity profiling, and whether or not the RNA helicases are involved in splicing.

Methods and compositions of the disclosure allow for characterization of selectivity of components of RNA metabolism including development of assays that typify the components such that inhibitors are sufficiently selective for their intended purpose. In particular embodiments, the disclosure encompasses RNA helicase and/or splicing regulator drug discovery systems, including by utilizing a biochemical platform comprising protein(s) for which an inhibitor is desired and one or more assays to monitor the in vitro activity of the protein. In specific embodiments, the protein to be targeted is an enzyme, and the protein may be a regulator (including an enzyme, in at least some cases) of splicing; in specific cases the protein is a component (whether permanent or transient) of the spliceosome. In specific embodiments, the disclosure encompasses use of a biochemical platform comprising purified, active RNA helicase proteins and assays to monitor their in vitro activity. Systems of the disclosure provide the assays required to evaluate target RNA helicase compound selectivity across a broader RNA helicase family. In some cases, the RNA helicases are involved in splicing, whereas in other cases the RNA helicases are involved in processes other than splicing, such as translation, rRNA biogenesis and processing, RNA decay, and so forth.

Although in some cases the methods concern selectivity profiling of RNA helicases that are not involved in splicing, in specific embodiments the methods concern selectivity profiling of inhibitors for RNA helicases that are involved in splicing or for other components related to splicing, such as splicing regulators of any kind. These splicing-related RNA helicases and other components may be directly involved in processing of the RNA such that they may be associated with cancer cells when they are defective, in some cases. As such, at least some methods of the disclosure provide for identification of inhibitors that target these splicing-related RNA helicases or splicing regulators for cancer cells. At the cellular level, use of the inhibitors may reduce the amount of misprocessed RNAs that had accumulated as a result of defective splicing component(s). The selectivity embodiment of the disclosure allows for identification of these inhibitors such that they also do not target RNA helicases or other components whose inhibition would be toxic to cells or an organism.

In particular embodiments, the disclosure provides improvements over methods in the art related to drug discovery of any kind by focusing evaluation of a particular inhibitor candidate with respect to a plurality of proteins, including a plurality of proteins of the same type or mechanism of action, but also with respect to ensuring counter selection against one or a plurality of proteins that may or may not be of the same type or mechanism of action. In a certain embodiment, the methods evaluate a particular inhibitor candidate with respect to a plurality of proteins involved in RNA metabolism but also that includes counter-selection against one or a plurality of proteins that are involved in RNA metabolism. In a specific embodiment, in vitro methods evaluate a particular inhibitor candidate with respect to RNA helicases or splicing regulators but also allowing counter-selectivity against RNA helicases or splicing regulators that are not desired to inhibited by the inhibitor candidate. Thus, the disclosure allows evaluation of compounds across diverse RNA helicases (“counter-selection”) to characterize and drive selectivity of small molecule inhibitors against a target RNA helicase of interest in vitro. The RNA helicases may or may not be involved in splicing, and in cases wherein the RNA helicase is not involved in splicing, in some cases it may be assessed by methods encompassed herein because they are structurally related to RNA helicases that are involved in splicing. In specific embodiments, the term structurally related refers to helicases that are of the same sub-family of helicases, e.g., DEAD-box or DEAH-box, RIG-I-like, Ski2-like, and SF1. The degree of similarity between subfamily members will be determined by the alignment score based on clustalw alignment, a pairwise based alignment that scores for the primary sequence similarity between two proteins. In some cases, a subset of helicases in a sub-family are similar in structure, including based on the aforementioned scoring.

Particular embodiments of the disclosure allow for characterizing and driving selectivity of RNA helicase inhibitors, for example during Lead Identification and Lead Optimization phases of drug discovery. Applications encompassed herein enable optimal development of screening assays and, at least in some cases, secondary assays of any kind during initial target feasibility phases of drug discovery and also optimization.

In particular embodiments, the present disclosure is directed to systems, methods, and compositions for analyzing compositions related to targeting RNA splicing and/or RNA helicases. Systems, methods, and compositions related to screening, characterization, and development of inhibitors of particular RNA splicing components, including at least RNA helicases, are encompassed herein.

Embodiments of the disclosure include screening for drug targets in RNA processing including screening for drug targets in RNA splicing. In specific embodiments, any screening method encompassed herein comprises characterization of one or more RNA helicases, including optimization of assays to ascertain functional activity of the one or more RNA helicases. In specific cases, an RNA helicase assay is optimized with respect to enzymatic activity such that subsequent screening steps produce accurate analyses of potential inhibition activity for one or more candidate inhibitors. Such optimization includes modifications of an enzymatic assay such as with respect to buffer type, buffer concentration, salts, concentration of ATP, presence or concentration of metals, a combination thereof, etc.

In some embodiments, one can utilize inhibitory small molecule fragments (typically less than 300 molecular weight) as an initial step to obtain inhibitory information and optimize an assay relevant to test for candidate inhibitors for the targets. Through screens and structure-activity relationship (SAR) methods, the inhibitory small molecular fragments can be analyzed to provide information on candidate inhibitors that may be tested, including for the ability to bind and inhibit the target. In cases where a structure of a small molecule fragments is determined to be useful for potential inhibition, one can employ medicinal chemistry to design, chemically synthesize, and/or develop a drug to be used as a pharmaceutical agent. Such information may be applied to other inhibitors for other RNA helicases as well, including other RNA helicases having similar RNA metabolism fingerprints (including RNA splicing fingerprints) upon inhibition. Additional assays include small molecule screens to identify chemical matter that binds to helicase(s) of interest and counterscreen against other helicases.

Embodiments of the disclosure encompass methods of screening for inhibitors in vitro for a plurality of RNA helicases, comprising the steps of: optionally optimizing conditions for an ATPase assay for each RNA helicase in the plurality; subjecting one or more candidate inhibitors to the ATPase assay for each RNA helicase in the plurality to identify candidate inhibitors that inhibit ATPase activity for a first subset of RNA helicases in the plurality but that do not inhibit ATPase activity for a second subset of RNA helicases in the plurality. In some cases, the subjecting step is further defined as: subjecting one or more candidate inhibitors to the ATPase assay for each RNA helicase in the plurality to identify candidate inhibitors that inhibit ATPase activity for a first subset of RNA helicases in the plurality, followed by identifying the absence of inhibition of ATPase activity for the second subset of RNA helicases in the plurality. In some cases, the subjecting step is further defined as: subjecting one or more candidate inhibitors to the ATPase assay for each RNA helicase in the plurality to identify candidate inhibitors that do not inhibit ATPase activity for the second subset of RNA helicases in the plurality, followed by identifying the presence of inhibition of ATPase activity for the first subset of RNA helicases in the plurality. In some cases, the subjecting step is further defined as: subjecting one or more candidate inhibitors to the ATPase assay for each RNA helicase in the plurality to identify candidate inhibitors that inhibit ATPase activity for a first subset of RNA helicases in the plurality at substantially the same time as identifying the absence of inhibition of ATPase activity for the second subset of RNA helicases in the plurality. In specific cases, the subjecting step comprises high throughput screening. In certain embodiments, the first subset of RNA helicases are of the same sub-family of helicases and/or the RNA helicases in the first subset of RNA helicases share the same function in RNA metabolism (such as RNA splicing).

In specific embodiments, candidate inhibitors may be of any type including at least small molecules, proteins, peptides, nucleic acid, carbohydrate, or a combination thereof.

In any methods encompassed herein, the method may utilize the plurality of RNA helicases that comprises two or more (including all or a majority) of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, cIF4A1, cIF4A2, cIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

In specific embodiments, any method of the disclosure further comprises analyzing the function of one or more RNA helicases in the plurality. The analyzing step may be performed in a cell or tissue or organism. Any analyzing step may be performed by a computer, including with an algorithm. In specific cases, the method further comprises analyzing activity of one or more candidate inhibitors in a cell, tissue, and/or organism. In a specific case, the analyzing comprises analyzing whether one or more candidate inhibitors inhibit one or more RNA helicases in the first subset but do not inhibit one or more RNA helicases in the second subset.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows a phylogenetic tree demonstrating the evolutionary relationships among RNA helicases based upon sequence similarity.

FIGS. 2A and 2B. FIG. 2A provides an example of parallelized assay development and structures. FIG. 2B provides an example of an output of an ATPase assay.

FIG. 3 illustrates a program for driving selectivity during hit optimization.

FIG. 4 shows one embodiment for a system of selectivity profiling against a class of RNA helicases.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment.” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z.” “(x and y) or z.” “x or (y and z),” or “x or y or z.” It is specifically contemplated that x, y, or z may be specifically excluded from an embodiment.

As used herein, the term “pharmacodynamic biomarker” or “PD biomarker” or “PD” refers to a biomarker whose level changes in response to exposure to an inhibitor (whether or not it is a test candidate inhibitor).

The term “platform” as used herein refers to collection of purified helicases with biochemical, associated assay(s) (ATPase, etc.) for one or more helicases or splicing regulators of interest. The total collection of assays, steps, stages, etc. may be used to determine the selectivity of one or more molecules. The nature of the assays, for example, may be determined by one or more factors, including helicase or splicing regulator of interest, inhibitor of interest, assay conditions, and so forth.

As used herein, the term “RNA splicing” refers to processing of RNA in which a newly made precursor messenger RNA transcript (often referred to as a “pre-mRNA”) is converted into a mature messenger RNA (mRNA). Such splicing includes removal of introns (non-coding regions) and linking of exons (coding regions). For many eukaryotic introns, a series of reactions catalyzed by the spliceosome produces the spliced mRNA.

As used herein, the term “selectivity” refers to the ability of an inhibitor to be able to inhibit the function of a desired component of RNA metabolism, such as an RNA helicase or splicing regulator, but wherein the inhibitor is not able to inhibit one or more other components of RNA metabolism, including one or more other RNA helicases or splicing regulators, respectively. In some cases, the selectivity comprises the ability to inhibit one or more particular, desired RNA helicases or splicing regulators but excludes the ability to inhibit one or more other RNA helicases or splicing regulators that would be toxic to a cell or organism if inhibited.

The term “splicing regulator” as used herein refers to any compound that directly or indirectly is associated with RNA splicing. The compound may be a protein, nucleic acid (e.g., small nuclear RNAs), and so forth. The splicing regulator may be a standing or transient component of the spliceosome. In particular cases, the splicing regulator (including when defective) is directly or indirectly associated with cancer, autoimmune disease, infectious disease, or neurodegeneration, such as the splicing regulator being in a defective state in cancer cells. In specific embodiments, the splicing regulator is one or more from the following list:

| Complex | Protein | Class/Family |
|---|---|---|
| Sm | SNRPB | Sm |
| Sm | SNRPD1 | Sm |
| Sm | SNRPD2 | Sm |
| Sm | SNRPD3 | Sm |
| Sm | SNRPE | Sm |
| Sm | SNRPF | Sm |
| Sm | SNRPG | Sm |
| U1 snRNP | RNU1-1 | U1 snRNP |
| U1 snRNP | SNRPA | U1 snRNP |
| U1 snRNP | SNRNP70 | U1 snRNP |
| U1 snRNP | SNRPC | U1 snRNP |
| U2 snRNP | RNU2-1 | 17S U2 snRNP |
| U2 snRNP | SNRPA1 | 17S U2 snRNP |
| U2 snRNP | SNRPB2 | 17S U2 snRNP |
| U2 snRNP | SF3B1 | 17S U2 snRNP |
| U2 snRNP | SF3B2 | 17S U2 snRNP |
| U2 snRNP | SF3B3 | 17S U2 snRNP |
| U2 snRNP | SF3B4 | 17S U2 snRNP |
| U2 snRNP | SF3B5 | 17S U2 snRNP |
| U2 snRNP | PHF5A | 17S U2 snRNP |
| U2 snRNP | SF3B6 | 17S U2 snRNP |
| U2 snRNP | SF3A1 | 17S U2 snRNP |
| U2 snRNP | SF3A2 | 17S U2 snRNP |
| U2 snRNP | SF3A3 | 17S U2 snRNP |
| U2 snRNP | DDX46 | 17S U2 snRNP associated |
| U2 snRNP | DDX39B | 17S U2 snRNP associated |
| U2 snRNP | HTATSF1 | 17S U2 snRNP associated |
| U5 snRNP | RNU5A-1 | U5 snRNP |
| U5 snRNP | SNRNP200 | U5 snRNP |
| U5 snRNP | PRPF8 | U5 snRNP |
| U5 snRNP | EFTUD2 | U5 snRNP |
| U5 snRNP | PRPF6 | U5 snRNP |
| U5 snRNP | DDX23 | U5 snRNP |
| U5 snRNP | CD2BP2 | U5 snRNP |
| U5 snRNP | SNRNP40 | U5 snRNP |
| U5 snRNP | TXNL4A | U5 snRNP |
| U4/U6 snRNP | RNU4-1 | U4/U6 snRNP |
| U4/U6 snRNP | RNU6-1 | U4/U6 snRNP |
| U4/U6 snRNP | PRPF4 | U4/U6 snRNP |
| U4/U6 snRNP | PRPF3 | U4/U6 snRNP |
| U4/U6 snRNP | PPIH | U4/U6 snRNP |
| U4/U6 snRNP | PRPF31 | U4/U6 snRNP |
| U4/U6 snRNP | NHP2L1 | U4/U6 snRNP |
| tri-snRNP | LSM2 | LSm |
| tri-snRNP | LSM3 | LSm |
| tri-snRNP | LSM4 | LSm |
| tri-snRNP | LSM5 | LSm |
| tri-snRNP | LSM6 | LSm |
| tri-snRNP | LSM7 | LSm |
| tri-snRNP | LSM8 | LSm |
| tri-snRNP | SART1 | tri-snRNP |
| tri-snRNP | USP39 | tri-snRNP |
| A complex | U2AF1 | 17S U2 snRNP associated |
| A complex | U2AF2 | 17S U2 snRNP associated |
| A complex | PUF60 | 17S U2 snRNP associated |
| A complex | SMNDC1 | 17S U2 snRNP associated |
| A complex | RBM17 | 17S U2 snRNP associated |
| A complex | U2SURP | 17S U2 snRNP associated |
| A complex | CHERP | 17S U2 snRNP associated |
| A complex | SF1 | A complex |
| A complex | PRPF40A | A complex |
| A complex | THRAP3 | A complex |
| A complex | RBM25 | A complex |
| A complex | CCAR1 | A complex |
| A complex | SUGP1 | A complex |
| A complex | RBM5 | A complex |
| A complex | RBM10 | A complex |
| A complex | HNRNPA1 | A complex |
| A complex | HNRNPAB | A complex |
| B complex | DHX15 | 17S U2 snRNP associated |
| PRP19 complex | PRPF19 | PRP19 complex |
| PRP19 complex | CDC5L | PRP19 complex |
| PRP19 complex | PLRG1 | PRP19 complex |
| PRP19 complex | CWC15 | PRP19 complex |
| PRP19 complex | BCAS2 | PRP19 complex |
| PRP19 complex | CTNNBL1 | PRP19 complex |
| PRP19 complex | WBP11 | PRP19 complex |
| PRP19 complex | PQBP1 | PRP19 complex |
| B complex | PPIE | PRP19 related |
| B complex | CRNKL1 | PRP19 related |
| B complex | SNW1 | PRP19 related |
| B complex | ISY1 | PRP19 related |
| B complex | XAB2 | PRP19 related |
| B complex | RBM22 | PRP19 related |
| B complex | PPIL1 | PRP19 related |
| B complex | BUD31 | PRP19 related |
| B complex | AQR | PRP19 related |
| B complex | SMU1 | B complex |
| B complex | MFAP1 | B complex |
| B complex | IK | B complex |
| B complex | WBP4 | B complex |
| B complex | TFIP11 | B complex |
| B complex | ZMAT2 | B complex |
| B complex | PRPF38A | B complex |
| B complex | PPIL4 | B complex |
| Bact complex | CWC27 | Bact complex |
| Bact complex | DHX16 | Bact complex |
| Bact complex | CWC22 | Bact complex |
| Bact complex | ZNF830 | Bact complex |
| Bact complex | CCDC12 | Bact complex |
| Bact complex | PPIL2 | Bact complex |
| Bact complex | GPKOW | Bact complex |
| Bact complex | RNF113A | Bact complex |
| Bact complex | PRCC | Bact complex |
| Bact complex | CWC25 | Bact complex |
| Bact complex | GPATCH1 | Bact complex |
| Bact complex | CDC40 | second step factor |
| Bact complex | BUD13 | RES complex |
| Bact complex | SNIP1 | RES complex |
| Bact complex | RBMX2 | RES complex |
| Bact complex | EIF4A3 | EJC |
| Bact complex | SAP18 | EJC |
| C complex | HSPA8 | PRP19 complex |
| C complex | SYF2 | C complex |
| C complex | DDX41 | C complex |
| C complex | CXorf56 | C complex |
| C complex | DGCR14 | C complex |
| C complex | C9orf78 | C complex |
| C complex | PPIL3 | C complex |
| C complex | PPWD1 | C complex |
| C complex | DHX35 | C complex |
| C complex | CACTIN | C complex |
| C complex | NOSIP | C complex |
| C complex | WDR83 | C complex |
| C complex | FAM50A | C complex |
| C complex | PPIG | C complex |
| C complex | SDE2 | C complex |
| C complex | CDK10 | C complex |
| C complex | LENG1 | C complex |
| C complex | FAM32A | C complex |
| C complex | FRA10AC1 | C complex |
| C complex | PRPF18 | second step factor |
| C complex | SLU7 | second step factor |
| C complex | DHX8 | second step factor |
| C complex | MAGOH | EJC |
| C complex | RBM8A | EJC |
| C complex | HNRNPC | hnRNP |
| C complex | SRRM2 | SR related |
| | SART3 | U4/U6 recycling |
| | DHX38 | second step factor |
| | LUC7L | U1 snRNP |
| | DDX42 | 17S U2 snRNP associated |
| | PRPF4B | B complex |
| | YJU2 | Bact complex |
| | RNPS1 | EJC |
| | ALYREF | EJC |
| | NXT1 | EJC |
| | NXF1 | EJC |
| | CASC3 | EJC |
| | ACIN1 | EJC |
| | UPF1 | EJC |
| | PNN | EJC |
| | PRPF38B | |
| | TCERG1 | |
| | SKIV2L2 | |
| | SNRNP27 | |
| | RUVBL1 | |
| | PRPF39 | |
| | MOV10 | |
| | GPATCH11 | |
| | C16orf80 | |
| | SRPK1 | |
| | SRPK2 | |
| | DBR1 | |
| | HNRNPUL1 | hnRNP |
| | FUS | hnRNP |
| | HNRNPA0 | hnRNP |
| | PCBP1 | hnRNP |
| | PCBP2 | hnRNP |
| | HNRNPA2B1 | hnRNP |
| | HNRNPA3 | hnRNP |
| | HNRNPAB | hnRNP |
| | HNRNPD | hnRNP |
| | HNRNPF | hnRNP |
| | RBMX | hnRNP |
| | HNRNPH1 | hnRNP |
| | HNRNPH3 | hnRNP |
| | HNRNPK | hnRNP |
| | HNRNPL | hnRNP |
| | HNRNPM | hnRNP |
| | HNRNPR | hnRNP |
| | HNRNPU | hnRNP |
| | RALY | hnRNP |
| | SYNCRIP | hnRNP |
| | HNRNPH2 | hnRNP |
| | HNRNPUL2 | hnRNP |
| | HNRNPDL | hnRNP |
| | RBMXL2 | hnRNP |
| | HNRNPCL1 | hnRNP |
| | SRSF1 | SR protein |
| | SRSF2 | SR protein |
| | SRSF4 | SR protein |
| | SRSF5 | SR protein |
| | SRSF6 | SR protein |
| | SRSF7 | SR protein |
| | SRSF11 | SR protein |
| | SRSF9 | SR protein |
| | SREK1 | SR protein |
| | TRA2B | SR protein |
| | TRA2A | SR protein |
| | SRSF3 | SR protein |
| | SFSWAP | SR protein |
| | SRSF12 | SR protein |
| | SRSF8 | SR protein |
| | SRSF10 | SR protein |
| | SRRM1 | SR related |

As used herein, the term “target” refers to a component of RNA metabolism that is desired to be inhibited. In specific embodiments, the target is an RNA helicase or splicing regulator that is desired to be inhibited specifically by one or more inhibitors. Any RNA helicase target may or may not be directly involved in splicing. In specific cases, the target is bound directly by the one or more inhibitors to effect the inhibition.

As used herein, the term “test inhibitor” or “candidate inhibitor” refers to a molecule that is being tested by one or more particular assays to identify targeting of a desired protein and also to identify absence of targeting of proteins not desired to be targeted by the inhibitor.

The present disclosure encompasses systems and methods that assess selectivity of any kind of inhibitor against any kind of RNA helicase.

In at least some cases, the present disclosure contemplates systems and methods that produce selective inhibitors of RNA metabolism, including at least RNA splicing; in specific cases, the selective inhibitors target one or more RNA helicases or splicing regulators but do not target other one or more RNA helicases or splicing regulators. The systems and methods utilize chemical, genetic, and/or computational means to characterize a plurality of RNA helicases to the extent that drug testing (through analysis of candidate inhibitors) identifies suitable inhibitors that selectively inhibit desired RNA helicases having common structure (for example) but are counter-selective for inhibiting RNA helicases that are not desired and, in at least some cases, may result in toxicity for a cell, tissue, or organism.

In some embodiments, the present disclosure concerns systems, methods, and compositions for targeting global RNA mis-splicing in cancer such that inhibitors are identified that prevent RNA mis-splicing that leads directly or indirectly to cancer. In certain embodiments, the disclosed embodiments allow for targeting a class of RNA helicases that are involved in RNA splicing, and in specific cases the RNA helicases are master regulators of RNA splicing. In particular embodiments, the RNA helicases are associated with defined patient indications at least in oncology, immuno-oncology, neurodegeneration, and so forth. The disclosed systems provide a biochemical platform that acts as a novel discovery engine for screening of and identification of selective RNA helicase inhibitors.

Embodiments of the disclosure include targeting of RNA splicing using one or more inhibitors identified in methods described herein, including RNA helicases, related to many disease indications, including oncology, immune-oncology, neurodegeneration, and so forth. In specific embodiments, inhibitors identified in methods encompassed herein are employed as pharmaceutical compositions for treatment of any medical condition in which defective RNA splicing is directly or indirectly related. The targeting may impact the function of the RNA helicase, including any biological complex that encompasses the helicase.

Certain embodiments allow for exploitation of defects in RNA splicing (including RNA helicases) to ascertain structure and/or function of one or more components in splicing to target any component of RNA splicing for treatment of any medical condition in which RNA splicing (including for RNA helicases) is directly or indirectly related.

### I. Biochemical Systems and Uses Thereof

The systems and methods encompassed herein include in vitro embodiments that screen biochemically for inhibitors of RNA metabolism components including RNA helicases, such as those that are involved in splicing.

To enable RNA helicase drug discovery programs, the present disclosure includes a biochemical platform comprising purified, active RNA helicase proteins and assays to monitor their in vitro activity. This platform provides the assays required to evaluate target RNA helicase compound selectivity across the broader RNA helicase family. The biochemical systems provide for in vitro evaluation of compounds across diverse RNA helicases (“counter-selection”) to characterize and drive selectivity of inhibitors of any kind, including small molecular inhibitors, against one or more target RNA helicases of interest.

The biochemical systems allow for characterization and driving of selectivity of RNA helicase inhibitors, including during all phases of drug discovery, such as lead identification and lead optimization. Such systems enable optimal development of screening assays and secondary assays during initial target feasibility phase of drug discovery.

In certain embodiments of the biochemical systems of the present disclosure, one can produce and assay activity of a plurality of RNA helicases. In specific cases, for a plurality of RNA helicases one can produce them as proteins in concordance with biochemical and/or biophysical assays for those proteins that would allow for measuring their activity or inhibition of activity. The inhibition of activity as described herein may be complete or may be partial, and either way such information may be informative for suitability of a particular candidate inhibitor.

Embodiments of the disclosure allow enablement during drug discovery to drive selectivity along with potency during drug discovery (candidate inhibitor testing), including during the development of inhibitors. This is in contrast to others in the related art that sequentially seek potency of candidate inhibitors before demonstrating selectivity of the inhibitors, in some cases.

Production of a plurality of RNA helicases for which an inhibitor is sought and their respective assay(s) for characterization, in specific embodiments, manifests as selectivity but also counter-selection for the inhibitor. In particular embodiments it is considered whether or not a candidate inhibitor, for example, can inhibit a particular helicase, and whether it can inhibit related RNA helicases, but also whether it can inhibit an essential protein (such as RNA helicases) and therefore would be undesirable. Thus, an inhibitor is sought that inhibits one or more desired RNA helicases but also does not inhibit one or more other RNA helicases. In such cases, an assay that determines selectivity may be the same type of assay that identifies counter-selectivity. In particular embodiments, if a candidate inhibitor inhibits an essential protein, such as a RNA helicase whose function is required for viability of a cell or tissue or organism, then the candidate inhibitor is no longer considered. Therefore, in at least some cases there is consideration of feasibility of the target and, when it is feasible, one can develop assays for that target.

Embodiments of the systems and methods of the disclosure include step(s) that include identification of one or more desired targets and optimization of one or more desired targets. In certain embodiments, a candidate inhibitor during multiple steps of a system or method are subjected to a counter-selection assay to facilitate selectivity for the candidate inhibitor. As part of this analysis, and/or prior to this analysis, assays may be individualized for a particular protein, such as for a potential ATPase activity. In at least some cases, a primary assay is an ATPase assay and conditions that work for one particular RNA helicase do not necessarily work for another RNA helicase such that optimization for each or multiple RNA helicases is necessary.

In some embodiments, a variety of assays may be available for determining the action of a particular protein, but methods of the disclosure require consideration of those assays that are suitable for a particular protein. In specific cases, there may be a large variety of methods for an ATPase assay, but the methods require consideration of ATPases that are suitable for a particular protein in question.

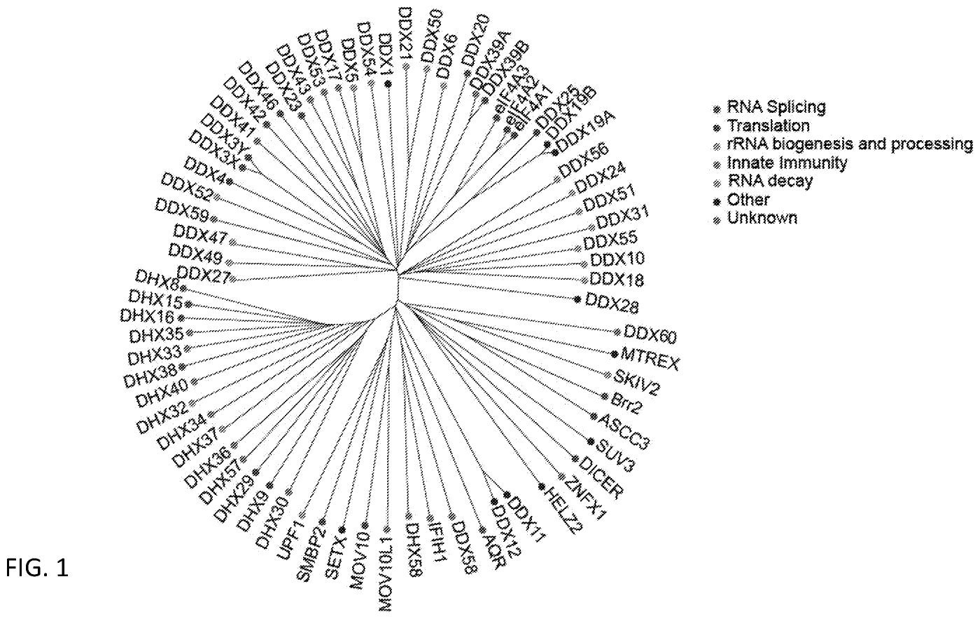

Embodiments of the biochemical system include screening for inhibitors for the group of proteins in FIG. 1. In specific embodiments, the group of proteins in FIG. 1 bind RNA, modify it in some manner, and also hydrolyze ATP, or in limited cases another nucleoside triphosphate, as a part of the modifying aspect. In specific aspects, one or more of the proteins in FIG. 1 have are related to RNA splicing associated directly or indirectly with cancer and other diseases. The disclosed methods and systems guide chemistry for the design of an inhibitor for a target but also counter-select against other unrelated targets. For example, if one tests a candidate inhibitor against the plurality of RNA helicases in FIG. 1, one can gather information about how to guide the chemistry to increase or continue to selectively inhibit a desired target yet counter-select against activity for the other targets.

In some embodiments one can subject a candidate inhibitor to one or more RNA helicases, including one or more RNA helicases shown in FIG. 1, and determine relative activity that inhibitor has on each of the tested RNA helicases to determine which helicases are being inhibited. This leads to understanding the success of the candidate inhibitor moving forward.

In particular embodiments, one or more of these RNA helicases are utilized as targets for medicine, including at least for oncology, immune-oncology, and neurodegeneration.

RNA helicases have diverse roles in RNA metabolism by participating in many steps of not only RNA splicing but other RNA processing pathways. They have been indicated to have roles in particular cancer indications, including at least breast, lung (non-small cell lung cancer and small cell lung cancer), pancreatic, colorectal, and acute myeloid leukemia (AML). The tractability of RNA helicases as targets in this system is attributed at least in part to ATPase and RNA binding functions that are required in cancer models and that are amenable to high throughput screening (HTS) assays.

The systems and methods of the disclosure exploit the tractability of RNA helicases as targets to develop inhibitors to the target helicases. This allows one to achieve an important aspect of the development of the inhibitors in the systems and methods herein, which is selectivity. The disclosure demonstrates how to produce effective (potent) and selective inhibitors by analyzing test candidate inhibitors in a manner to identify inhibitors that are selective for inhibiting one or more desired target proteins yet that are unable to target one or more target proteins that are not intended to be targeted. In particular embodiments, the inhibitors are selective for their one or more particular targets while excluding others by targeting the mechanism of action of their one or more particular targets. In at least some cases, the inhibitors are selective for their one or more particular targets while excluding others because those proteins for which they are able to target all share the same or a similar mechanism of action.

The following includes a list of members of the phylogenic tree of FIG. 1: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, cIF4A1, cIF4A2, cIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27. Any one or two or more of the proteins listed in FIG. 1 may be an RNA helicase that is a target for screening for an inhibitor. In some cases, one or more of these are not to be targeted because their inhibition would be toxic to a cell, tissue, or organism.

In particular embodiments for the plurality of RNA helicases listed in FIG. 1, there is a subset of the plurality that is suitable for targeting because they are non-essential (not toxic when inhibited) and there is another subset of the plurality that is not suitable for targeting because it is essential (toxic when inhibited). The plurality of multiple RNA helicases that are suitable for targeting may or may not be in the same subset because they are structurally related, such as having a certain percentage identity between two RNA helicases or having a certain percentage identity among more than two RNA helicases. The plurality of multiple RNA helicases that are suitable for targeting may or may not be in the same subset because they are functionally related.

### II. ATPase Assay

In particular embodiments, a candidate inhibitor for inhibiting one or more RNA helicases is assayed by measuring its ability to impact the functionality of the RNA helicase, including enzymatic activity, such as ATPase activity of the RNA helicase. Thus, embodiments of the present systems and methods include ATPase assays. In particular embodiments of ATPase assays, phosphate release in the presence of ATP is measured, and the output of the assay may be of any suitable kind including at least a colorimetric, fluorescent, or radioactive output based on corresponding respective substrates.

In some cases, the ATPase assay is optimized for a particular one or more RNA helicases such that the ATPase provides an effective measure of the ATPase activity and kinetics of the respective RNA helicase(s). Such optimization may be of any kind, including adjustment of buffer concentrations, buffer pHs, buffer composition, salts, concentration and type of NTP, presence or concentration of one or more particular metals, a combination thereof, etc. Different RNA species, i.e., polyA, polyG, or more specific RNA sequences to be determined per helicase, may stimulate activity of different helicases to different degrees. Additionally the reaction time and temperature dependences may differ between helicases. In some cases, each protein in a plurality of RNA helicases is optimized in the ATPase assay.

In some embodiments, one can measure IC50 of one or more inhibitors and measure the Km for ATP and RNA substrates and the k_{cat} of the enzymes.

### III. Inhibitors

The systems and methods of the disclosure include in vitro screens related to identifying inhibitors of RNA helicases or splicing regulators, including for clinical purposes. The inhibitors being assayed in the disclosed systems and methods are candidate inhibitors while determination is made as to their suitability for selective inhibition of RNA helicases or splicing regulators. A candidate inhibitor that selectively inhibits desired one or more RNA helicases or splicing regulators but that does not inhibit other RNA helicases or splicing regulators that are essential is an inhibitor that may be applied for clinical applications.

The inhibitors may be obtained from any source, including generated de novo or obtained from a library, whether commercial or not. The inhibitors may be selected for having one or more structural or functional attributes, such as similarity to other known RNA helicase inhibitors or based on structure-activity relationship analysis. In other cases, one or more attributes for the inhibitors include a structural component that is known or suspected of being useful to inhibit functionality of certain enzymatic mechanisms of action. In other cases, the inhibitors are selected from a library without any knowledge of useful attributes for the inhibitors.

In some embodiments, the inhibitor may be any type of inhibitor and any type of molecule. Although in particular embodiments the inhibitor is a small molecule, in alternative embodiments the inhibitor is not a small molecule, such as a protein, peptide, nucleic acid, carbohydrate, or a combination thereof. The term “small molecule” as used herein refers to an organic compound having a size of less than 1500 Daltons.

These compounds may be competitive with the NTP site, competitive with the RNA binding site, competitive with an accessory protein binding site, bind to an allosteric site to block access to one or both of the NTP and RNA binding sites, enhance binding to preclude hydrolysis of the NTP, enhance binding but restrict the processivity of across the RNA species, or have another allosteric function. In some cases, an inhibitor inhibits an RNA helicase by inhibiting a site or function other than with respect to its enzymatic activity. For example, an inhibitor can inhibit binding to a RNA substrate, binding to another regulator protein, or a combination thereof.

### IV. Methods of Treatment

In particular embodiments, the methods encompassed herein identify one or more compounds that are useful for treatment of any medical condition. In some cases, the medical condition is directly or indirectly treated based on administration of one or more compounds identified herein. In some cases, the medical condition is treated by a compound that directly or indirectly impacts RNA metabolism, including RNA processing of any kind. In specific cases, the medical condition is treated by a compound that targets an RNA helicase or splicing inhibitor, either through inhibition or activation. That is, in some cases a compound identified by methods herein inhibits a defective RNA helicase that without the inhibition produces an accumulation of misprocessed RNA associated with cancer. In some cases a compound identified by methods herein inhibits an RNA helicase that then increases accumulation of certain misprocessed RNA that activates antitumor signaling and/or antiviral signaling. In any event, a therapeutically effective amount of one or more inhibitors screened from or otherwise identified by methods disclosed herein may be provided to an individual in need thereof. In some cases, the medical condition includes cancer of any kind, autoimmune disease, infectious disease, neurodegeneration, and so forth.

Any cancers disclosed herein may have defective splicing for which the inhibitors identified by methods encompassed herein are therapeutic for the cancer. In some embodiments, an RNA helicase is targeted as being associated with aberrant splicing because the RNA helicase is mutated, and the mutation may be the direct or indirect cause of the medical condition. Regardless of action, in some embodiments, the inhibitors identified through screens herein are useful for alleviation of at least one symptom in cancer, in specific cases. The cancer may be of any type or grade or tissue of origin. It may or may not be metastatic. Tumors for which the inhibitors identified through methods disclosed herein are useful include any malignant cell type, such as those found in a solid tumor or a hematological tumor. In some cases, the inhibitor targets one or more defective RNA helicases that are associated with chemo-refractory malignancies. Specific cancers for which the inhibitors identified through methods disclosed herein are useful include non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, and so on. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In certain embodiments, compounds identified by screening or other methods encompassed herein are provided in a therapeutically effective amount to an individual with an autoimmune disease. In specific cases, the autoimmune disease is the result directly or indirectly of aberrant splicing. In particular embodiments, the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, Dermatomyositis, and so forth.

In certain embodiments, the compounds identified by screening or other methods encompassed herein are provided in a therapeutically effective amount to an individual with neurodegeneration, such as associated with neurodegenerative diseases including at least amyotrophic lateral sclerosis, multiple sclerosis, Parkinson's disease, Alzheimer's disease, Huntington's disease, and prion diseases.

In some embodiments, the compounds identified by screening or other methods encompassed herein are provided in a therapeutically effective amount to an individual with an infectious disease. The infectious disease may be of any kind, including at least bacterial, viral, fungal, or parasitic.

Examples of viruses associated with infectious disease include, but are not limited to, at least adenovirus, alphavirus, calicivirus, coronavirus (including SARS CoV2 and SARS CoV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus. Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, and so forth.

Examples of bacteria associated with infectious disease include, but are not limited to, at least Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaca, Salmonella, Shigella, Staphylococcus, Streptococcus, Treponema, and Yersinia.

Examples of fungus associated with infectious disease include, but are not limited to, at least Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paccilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha.

Examples of protozoa associated with infectious disease include, but are not limited to, at least Acanthocheilonema, Aclurostrongylus, Ancylostoma, Angiostrongylus, Ascaris, Babesia, Balantidium, Besnoitia, Brugia, Bunostomum, Capillaria, Chabertia, Cooperia, Crenosoma, Cryptosporidium, Dictyocaulus, Dioctophyme, Dipetalonema, Diphyllobothrium, Diplydium, Dirofilaria, Dracunculus, Enterobius, Eimeria, Encephalitozoon, Entamoeba, Filaroides, Giardia, Haemonchus, Hammondia, Hepatozoon, Isospora, Lagochilascaris, Leishmania, Loa, Mansonella, Microsporidia, Muellerius, Nanophyetus, Necator, Nematodirus, Neospora, Nosema, Oesophagostomum, Onchocerca, Opisthorchis, Ostertagia, Parafilaria, Paragonimus, Parascaris, Pentatrichomonas, Physaloptera, Plasmodium, Pneumocystis, Protostrongylus, Sarcocystis, Schistosoma, Setaria, Spirocerca, Spirometra, Stephanofilaria, Strongyloides, Strongylus, Theileria, Thelazia, Toxascaris, Toxocara, Toxoplasma, Trichinella, Trichostrongylus, Trichuris, Trypanosoma, Uncinaria, and Wuchereria.

Compounds identified from methods encompassed herein may be formulated specifically for therapeutic use. In specific cases the inhibitor identified by screening methods and symptoms herein is formulated in a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In specific embodiments, the disclosure encompasses methods of treating a subject (including a mammal such as a human) having a medical condition associated with defective RNA metabolism comprising administering to the subject a pharmaceutical composition comprising a therapeutically effective amount of a selective inhibitor of one or more RNA helicases identified by any method herein. In specific embodiments there are methods of treating a subject having a medical condition associated with defective RNA metabolism comprising administering to the subject a pharmaceutical composition comprising a therapeutically effective amount of a selective inhibitor of one or more RNA helicases wherein the inhibitor is identified by an in vitro screening method comprising the steps of: optionally optimizing conditions for an ATPase assay for an RNA helicase of a plurality of RNA helicases and subjecting a test candidate to the ATPase assay for each RNA helicase in the plurality to identify the selective inhibitor that inhibits ATPase activity for a first subset of RNA helicases in the plurality but that do not inhibit ATPase activity for a second subset of RNA helicases in the plurality. The defective RNA metabolism may be of any kind, including defective synthesis, folding/unfolding, modification, processing, stability, or degradation of RNA; accumulation of misprocessed RNA; defective RNA splicing, or a combination thereof. In specific embodiments, the subjecting step is further defined as subjecting one or more test candidates to the ATPase assay for each RNA helicase in the plurality to identify test candidates that inhibit ATPase activity for a first subset of RNA helicases in the plurality, followed by identifying the absence of inhibition of ATPase activity for the second subset of RNA helicases in the plurality. In specific embodiments, the subjecting step is further defined as subjecting one or more test candidates to the ATPase assay for each RNA helicase in the plurality to identify test candidates that do not inhibit ATPase activity for the second subset of RNA helicases in the plurality, followed by identifying the presence of inhibition of ATPase activity for the first subset of RNA helicases in the plurality. In some embodiments, the subjecting step is further defined as subjecting one or more test candidates to the ATPase assay for each RNA helicase in the plurality to identify test candidates that inhibit ATPase activity for a first subset of RNA helicases in the plurality at substantially the same time as identifying the absence of inhibition of ATPase activity for the second subset of RNA helicases in the plurality. Any subjecting step may comprise high throughput screening. In certain embodiments, the first subset of RNA helicases (which may comprise DHX15) is of the same sub-family of helicases or the RNA helicases in the first subset of RNA helicases share the same function (such as splicing) in RNA metabolism

In such cases, the test candidates may be small molecules, proteins, peptides, nucleic acid, carbohydrate, or a combination thereof.

In some embodiments, the plurality of RNA helicases comprises two or more, including all in some cases, of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, CIF4A1, cIF4A2, cIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

In some embodiments, the method further comprises analyzing the function of one or more RNA helicases in the plurality, such as being performed in a cell or tissue or organism or performed by a computer. The method may further comprise analyzing activity of one or more test candidates in a cell, tissue, and/or organism or further comprises analyzing activity of one or more test candidates by a computer. The analyzing may comprise analyzing whether one or more test candidates inhibit one or more RNA helicases in the first subset but do not inhibit one or more RNA helicases in the second subset.

In specific embodiments, the medical condition being treated is cancer, including a solid tumor or a hematological tumor. The inhibitor may target one or more defective RNA helicases that are associated with chemo-refractory malignancies. The cancer may be selected from the group consisting of non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma. The cancer may be a histological type comprising neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

In some embodiments, the medical condition being treated is an autoimmune disease, including one that is a result of direct or indirect aberrant splicing. In specific embodiments, the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, and Dermatomyositis.

In some embodiments, the medical condition being treated is a neurodegenerative disease, such as one selected from the group consisting of amyotrophic lateral sclerosis, multiple sclerosis, Parkinson's disease, Alzheimer's disease, Huntington's disease, and prion diseases.

In some embodiments, the medical condition is an infectious disease, including an infectious disease that is at least bacterial, viral, fungal, or parasitic. The infectious disease may be selected from the group consisting of adenovirus, alphavirus, calicivirus, coronavirus (including SARS CoV2 and SARS COV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus. Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), and adenovirus.

The pharmaceutical composition utilized in the method may further comprise a pharmaceutically acceptable carrier, including one that is selected from the group consisting of solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and any combinations thereof. The pharmaceutically composition may be administered in solid, liquid or aerosol form. The pharmaceutically composition may need to be sterile for administration as injection. The pharmaceutically composition may be administered by any suitable method, including intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by any combinations thereof.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Biochemical Platform for Screening for Selective RNA Helicase Inhibitors

FIG. 1 shows a phylogenetic tree demonstrating the evolutionary relationships among RNA helicases based upon sequence similarity. In some embodiments, selective inhibitors of one or more of these specific RNA helicase targets may be developed via parallelized biochemical assays. In FIG. 1, for example, there may be parallelized target protein production wherein one can at substantially the same time target protein production that allows prioritization of helicases that are (a) targets; (b) related to targets; or (c) are toxic helicases that are not desired to be targeted by any inhibitor. In specific examples, 15 RNA helicases have been vetted, and these include at least the following: Brr2, cIF4A1, cIF4A2, cIF4A3, DHX8, DHX15, DHX38, DHX40, DDX21, DDX19A, DDX19B, DDX39A, DDX39B, DDX25, and DDX52.

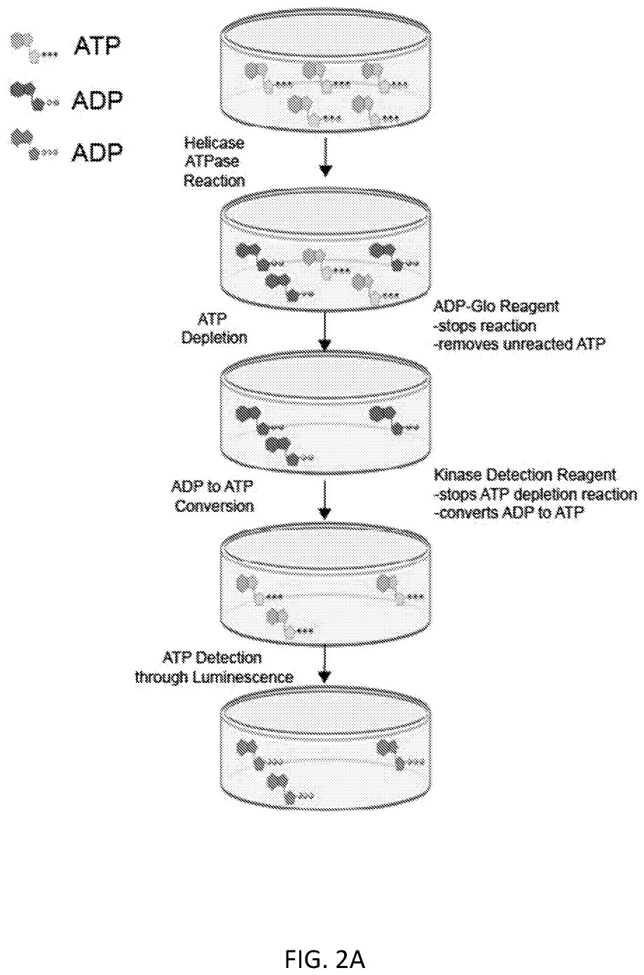

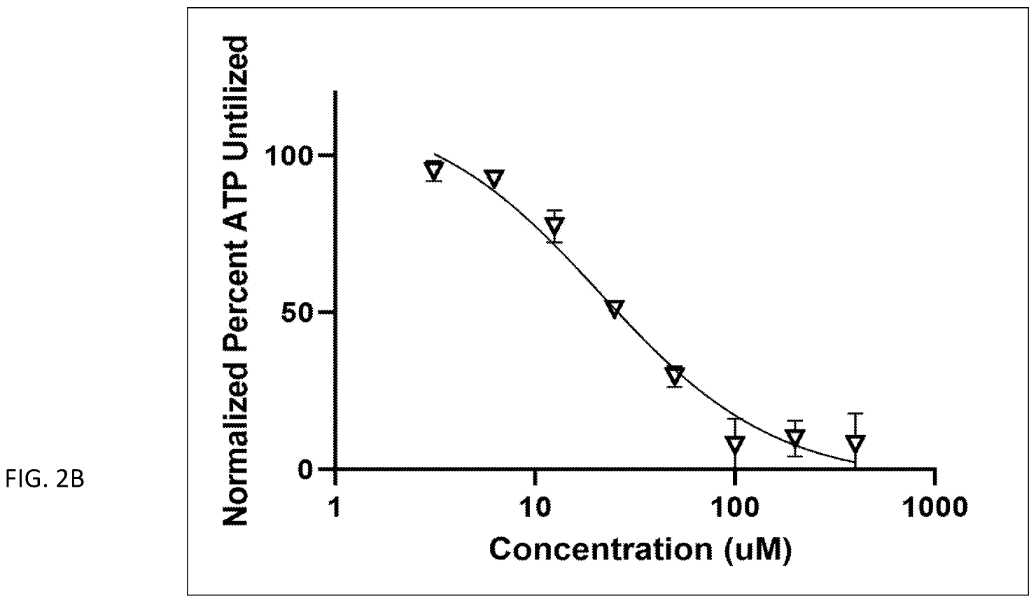

FIG. 2 provides one example of parallelized assay development and structures. Embodiments of the disclosure include development of standardized ATPase assays as primary assays across a plurality of helicases. Additional assays may be employed, for example for verification, such as nucleotide- and RNA-probe fluorescence polarization (FP) assays.

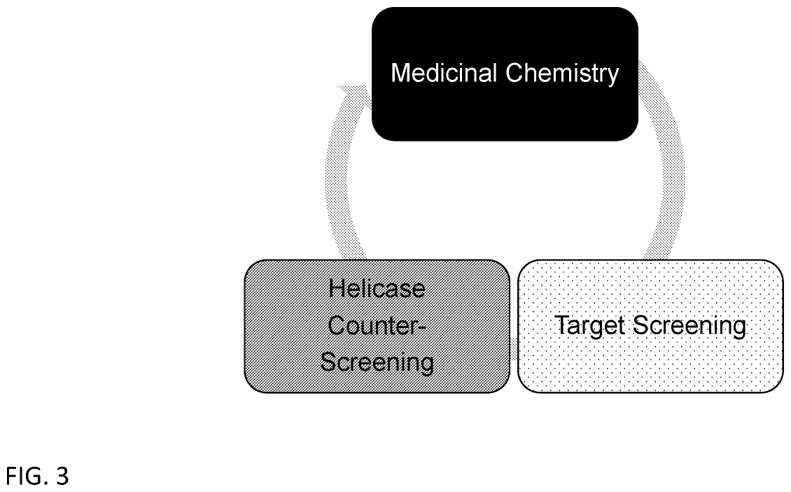

FIG. 3 illustrates a program for driving selectivity during hit optimization. The embodiment allows for target screening to drive selectivity early in the process so as to engineer out essential RNA helicases upon helicase counter-screening. The resultant output is used at least in part as a basis for medicinal chemistry practices to produce and characterize an inhibitor that is sufficiently effective and sufficiently selective to be used in a clinical setting. The information in this process is useful to characterize potential targets in oncology, immune-oncology, and other indications, such as immunology.

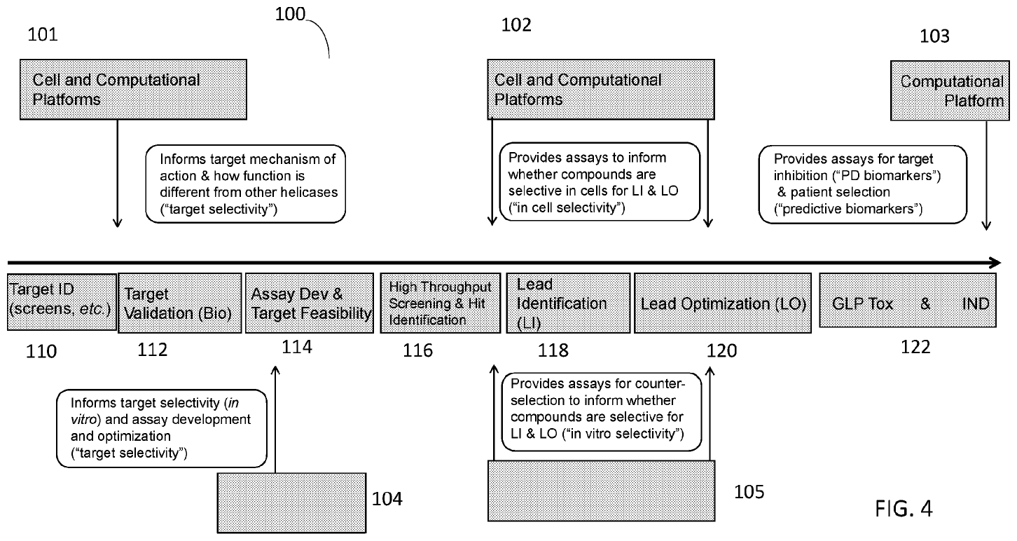

FIG. 4 provides one example of a system 100 for developing or identifying compounds useful for manipulating processes associated with RNA metabolism. In specific cases, the system 100 is utilized for identifying or screening for inhibitors against a class of proteins related to RNA metabolism, such as RNA helicases or splicing regulators, in which the system includes selectivity profiling throughout multiple steps or stages, in at least some cases. Although in specific embodiments the order of steps or actions are successive in nature from left to right of the image, in alternative embodiments the order of steps or actions are modified compared to this order. For example, one step or action may occur before a subsequent step or action as depicted in FIG. 4, but in alternative embodiments an order of succession is reversed. In some cases one or more steps or actions may occur at substantially the same time. In particular embodiments, FIG. 4 illustrates a system 100 for identifying inhibitors of RNA helicases and/or splicing regulators in which the respective RNA helicases and/or splicing regulators are selectively profiled so that they target one or more particular helicases and/or splicing regulators but also that they do not target other proteins, including other respective helicases and/or splicing regulators. Such an aspect of the system may be utilized because inhibition of the excluded helicases and/or splicing regulators would be toxic to a cell or tissue or organ or individual. In particular embodiments, the system of the present disclosure is an in vitro system.

In a specific example of an embodiment of the system 100 of FIG. 4, the initial one or two steps or actions incorporate cellular and/or computational platforms 101 that inform for a particular one or more helicases a target mechanism of action and/or how the one or more particular helicases function differently from others, and this may be considered target selectivity. Such initial one or two steps or actions may include target identification 110, such as by screens, and so forth. The target identification 110 may identify one target, or a plurality of related targets, for which inhibition may be desirable, such as may be useful for a clinical application. The screens may assay for one or more structural characteristics and/or one or more functional characteristics of one or a plurality of potential target proteins, but in specific embodiments the assay provides information at least on a target mechanism of action. In particular embodiments, target identification 110 comprises disruption of one or more RNA helicases and/or one or more splicing regulators individually or as a pool and analysis of the pertinent normal or cancer cell phenotype(s) which is in some instances growth. In some cases, disruption of one or more RNA helicases and/or one or more splicing regulators has no substantive impact on growth, and this may or may not be informative. In other cases, disruption of one or more RNA helicases and/or one or more splicing regulators enhances or impairs growth. The pertinent phenotype may be measured by any suitable methods, including cell counts, for example. Following target identification 110, there may be a target validation step 112 in which the functional and/or structural characteristics of the identified targets in target identification step 110 are validated through biological means. In some cases, biological validation of a particular target or targets includes target perturbation followed by cell growth, cell death, cell signaling and/or RNA-based and protein-based measurements to discern target mechanism of action in the appropriate cellular context in vitro or in vivo. These studies provide further evidence that target inhibition may be desirable in a particular clinical context and provide information that may be utilized as pharmacodynamic markers of target inhibition.

Following one to two steps or actions utilizing cell and computational platforms, the next one or more steps may implement biochemical platform 104 in which information about target selectivity in vitro is obtained and target selectivity may be maintained for assay development and optimization or feasibility step 114 for one or a plurality of target proteins, including RNA helicases and/or splicing regulators. Thus, in this stage as well, target selectivity is performed, in certain embodiments. As a result of the assay development and optimization of feasibility step 116, there may or may not be high throughput screening (HTS) for a plurality of test candidates that inhibit one or more desired proteins. The test candidates that show favorable characteristics in the HTS will be identified. These characteristics include tractable chemistry, inhibition of activity when treated with 10 uM, and no discernable chemical liabilities.

The test candidate inhibitors that are the outcome of steps 110, 112, 114, and 116 may be considered lead compound(s) and may be subject to additional cell and computational platforms 102 and biochemical platform 105 for enhanced characterization. The cell and computational platforms 102 provide assays to obtain information whether compounds are selective in cells. The biochemical platform 105, which may occur at substantially the same time as cell and computational platforms 102, may provide in vitro assays for counter-selection to provide information whether the compounds are selective or maintain selectivity. Following this, the lead compound(s) may be subject to computational platform 103 in which assays are utilized for target inhibition to identify PD biomarkers and/or to identify predictive biomarkers for selection of recipient individuals for which the lead compound(s) would be therapeutically effective.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of screening for inhibitors in vitro for a plurality of RNA helicases, comprising the steps of:
optionally optimizing conditions for an ATPase assay for each RNA helicase in the plurality;
subjecting one or more candidate inhibitors to the ATPase assay for each RNA helicase in the plurality to identify candidate inhibitors that inhibit ATPase activity for a first subset of RNA helicases in the plurality but that do not inhibit ATPase activity for a second subset of RNA helicases in the plurality.

2. The method of claim 1, wherein the subjecting step is further defined as:
subjecting one or more candidate inhibitors to the ATPase assay for each RNA helicase in the plurality to identify candidate inhibitors that inhibit ATPase activity for a first subset of RNA helicases in the plurality, followed by identifying the absence of inhibition of ATPase activity for the second subset of RNA helicases in the plurality.

3. The method of claim 1, wherein the subjecting step is further defined as:
subjecting one or more candidate inhibitors to the ATPase assay for each RNA helicase in the plurality to identify candidate inhibitors that do not inhibit ATPase activity for the second subset of RNA helicases in the plurality, followed by identifying the presence of inhibition of ATPase activity for the first subset of RNA helicases in the plurality.

4. The method of claim 1, wherein the subjecting step is further defined as:
subjecting one or more candidate inhibitors to the ATPase assay for each RNA helicase in the plurality to identify candidate inhibitors that inhibit ATPase activity for a first subset of RNA helicases in the plurality at substantially the same time as identifying the absence of inhibition of ATPase activity for the second subset of RNA helicases in the plurality.

5. The method of any one of the preceding claims, wherein the candidate inhibitors are small molecules, proteins, peptides, nucleic acid, carbohydrate, or a combination thereof.

6. The method of claim 5, wherein the candidate inhibitors are small molecules.

7. The method of any one of the preceding claims, wherein the subjecting step comprises high throughput screening.

8. The method of any one of the preceding claims, wherein the first subset of RNA helicases are of the same sub-family of helicases.

9. The method of any one of the preceding claims, wherein the RNA helicases in the first subset of RNA helicases share the same function in RNA metabolism.

10. The method of claim 10, wherein the function is RNA splicing.

11. The method of any one of the preceding claims, wherein the plurality of RNA helicases comprises two or more of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, CIF4A1, cIF4A2, cIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

12. The method of claim 11, wherein the plurality of RNA helicases comprises all of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, CIF4A1, cIF4A2, cIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

13. The method of any one of the preceding claims, wherein the plurality of RNA helicases comprises DHX15.

14. The method of any one of the preceding claims, wherein the first subset of RNA helicases comprises DHX15.

15. The method of any one of the preceding claims, wherein the method further comprises analyzing the function of one or more RNA helicases in the plurality.

16. The method of claim 15, wherein the analyzing step is performed in a cell or tissue or organism.

17. The method of claim 15 or 16, wherein the analyzing step is performed by a computer.

18. The method of any one of the preceding claims, wherein the method further comprises analyzing activity of one or more candidate inhibitors in a cell, tissue, and/or organism.

19. The method of any one of the preceding claims, wherein the method further comprises analyzing activity of one or more candidate inhibitors by a computer.

20. The method of claim 18 or 19, wherein the analyzing comprises analyzing whether one or more candidate inhibitors inhibit one or more RNA helicases in the first subset but do not inhibit one or more RNA helicases in the second subset.

21. A method of treating a subject having a medical condition associated with defective RNA metabolism comprising administering to the subject a pharmaceutical composition comprising a therapeutically effective amount of a selective inhibitor of one or more RNA helicases, wherein the inhibitor is identified by an in vitro screening method comprising the steps of:
optionally optimizing conditions for an ATPase assay for an RNA helicase of a plurality of RNA helicases and
subjecting a test candidate to the ATPase assay for each RNA helicase in the plurality to identify the selective inhibitor that inhibits ATPase activity for a first subset of RNA helicases in the plurality but that do not inhibit ATPase activity for a second subset of RNA helicases in the plurality.

22. The method of claim 21, wherein the defective RNA metabolism is defective synthesis, folding/unfolding, modification, processing, stability, or degradation of RNA.

23. The method of claim 21 or 222, wherein the defective RNA metabolism is accumulation of misprocessed RNA.

24. The method of any one of claims 21-223, wherein the defective RNA metabolism is defective RNA splicing.

25. The method of claim 21, wherein the subjecting step is further defined as:
subjecting one or more test candidates to the ATPase assay for each RNA helicase in the plurality to identify test candidates that inhibit ATPase activity for a first subset of RNA helicases in the plurality, followed by identifying the absence of inhibition of ATPase activity for the second subset of RNA helicases in the plurality.

26. The method of claim 21, wherein the subjecting step is further defined as:
subjecting one or more test candidates to the ATPase assay for each RNA helicase in the plurality to identify test candidates that do not inhibit ATPase activity for the second subset of RNA helicases in the plurality, followed by identifying the presence of inhibition of ATPase activity for the first subset of RNA helicases in the plurality.

27. The method of claim 21, wherein the subjecting step is further defined as:
subjecting one or more test candidates to the ATPase assay for each RNA helicase in the plurality to identify test candidates that inhibit ATPase activity for a first subset of RNA helicases in the plurality at substantially the same time as identifying the absence of inhibition of ATPase activity for the second subset of RNA helicases in the plurality.

28. The method of any one of claims 21-227, wherein the test candidates are small molecules, proteins, peptides, nucleic acid, carbohydrate, or a combination thereof.

29. The method of claim 228, wherein the test candidates are small molecules.

30. The method of any one of claims 21-229, wherein the subjecting step comprises high throughput screening.

31. The method of any one of claims 21-30, wherein the first subset of RNA helicases is of the same sub-family of helicases.

32. The method of any one of claims 21-31, wherein the RNA helicases in the first subset of RNA helicases share the same function in RNA metabolism.

33. The method of claim 32, wherein the function is RNA splicing.

34. The method of any one of claims 21-33, wherein the plurality of RNA helicases comprises two or more of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

35. The method of claim 34, wherein the plurality of RNA helicases comprises all of the following RNA helicases: DHX8, DHX15, DHX16, DHX35, DHX33, DHX38, DHX40, DHX32, DHX34, DHX37, DHX36, DHX57, DHX29, DHX9, DHX30, UPF1, SMBP2, SETX, MOV10, MOV10L1, DHX58, IFIH1, DDX58, AQR, DDX12, DDX11, HELZ2, ZNFX1, DICER, SUV3, ASCC3, Brr2, SKIV2, MTREX, DDX60, DDX28, DDX18, DDX10, DDX55, DDX31, DDX51, DDX24, DDX56, DDX19A, DDX19B, DDX25, eIF4A1, eIF4A2, eIF4A3, DDX39B, DDX39A, DDX20, DDX6, DDX50, DDX21, DDX1, DDX54, DDX5, DDX17, DDX53, DDX43, DDX23, DDX46, DDX42, DDX41, DDX3Y, DDX3X, DDX4, DDX52, DDX59, DDX47, DDX49, and DDX27.

36. The method of any one of claims 21-35, wherein the plurality of RNA helicases comprises DHX15.

37. The method of any one of claims 21-36, wherein the first subset of RNA helicases comprises DHX15.

38. The method of any one of claims 21-37, wherein the method further comprises analyzing the function of one or more RNA helicases in the plurality.

39. The method of claim 38, wherein the analyzing step is performed in a cell or tissue or organism.

40. The method of claim 38 or 39, wherein the analyzing step is performed by a computer.

41. The method of any one of claims 21-40, wherein the method further comprises analyzing activity of one or more test candidates in a cell, tissue, and/or organism.

42. The method of any one of claims 21-41, wherein the method further comprises analyzing activity of one or more test candidates by a computer.

43. The method of claim 41 or 42, wherein the analyzing comprises analyzing whether one or more test candidates inhibit one or more RNA helicases in the first subset but do not inhibit one or more RNA helicases in the second subset.

44. The method of any one of claims 21-43, wherein the medical condition is cancer.

45. The method of any one of claims 21-44, wherein the cancer is a solid tumor or a hematological tumor.

46. The method of any one of claims 21-45, wherein the inhibitor targets one or more defective RNA helicases that are associated with chemo-refractory malignancies.

47. The method of any one of claims 21-46, wherein the cancer is selected from the group consisting of non-small cell lung cancer adenocarcinoma, ovarian cancer, esophageal cancer, HCC, head and neck cancer, non-small cell lung squamous cancer, breast cancer (including at least triple-negative), gastric cancer, pancreatic cancer, bladder cancer, colon cancer, cecum cancer, stomach cancer, brain cancer, kidney cancer, larynx cancer, sarcoma, lung cancer, melanoma, prostate cancer, tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

48. The method of any one of claims 21-47, wherein the cancer is a histological type comprising neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

49. The method of any one of claims 21-48, wherein the medical condition is an autoimmune disease.

50. The method of any one of claims 21-49, wherein the autoimmune disease is a result of direct or indirect aberrant splicing.

51. The method of any one of claims 21-50, wherein the autoimmune disease is selected from the group consisting of Type 1 diabetes, rheumatoid arthritis, psoriasis, multiple sclerosis, Systemic lupus erythematosus, Graves' disease, inflammatory bowel disease, Addison's disease, Sjögren's syndrome, Hashimoto's thyroiditis, Myasthenia gravis, celiac disease, Autoimmune vasculitis, Pernicious anemia, and Dermatomyositis.

52. The method of any one of claims 21-51, wherein the medical condition is a neurodegenerative disease.

53. The method of any one of claims 21-52, wherein the neurodegenerative disease is selected from the group consisting of amyotrophic lateral sclerosis, multiple sclerosis, Parkinson's disease, Alzheimer's disease, Huntington's disease, and prion diseases.

54. The method of any one of claims 21-53, wherein the medical condition is an infectious disease.

55. The method of any one of claims 21-54, wherein the infectious disease is at least bacterial, viral, fungal, or parasitic.

56. The method of any one of claims 21-55, wherein the infectious disease is selected from the group consisting of adenovirus, alphavirus, calicivirus, coronavirus (including SARS CoV2 and SARS COV), distemper virus, Ebola virus, enterovirus, flavivirus, hepatitis virus, herpesvirus (including herpes simplex virus or varicella zoster virus), infectious peritonitis virus, influenza virus, leukemia virus, Marburg virus, orthomyxovirus, papilloma virus, parainfluenza virus, paramyxovirus, parvovirus, pestivirus, picorna virus, pox virus, rabies virus, reovirus, retrovirus, and rotavirus. Specific viruses include at least human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), and adenovirus.

57. The method of any one of claims 21-56, wherein the pharmaceutical composition further comprises a pharmaceutically acceptable carrier.

58. The method of any one of claims 21-57, wherein the pharmaceutically acceptable carrier is selected from the group consisting of solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and any combinations thereof.

59. The method of any one of claims 21-58, wherein the pharmaceutically composition is administered in solid, liquid or aerosol form.

60. The method of any one of claims 21-59, wherein the pharmaceutically composition needs to be sterile for administration as injection.

61. The method of any one of claims 21-60, wherein the pharmaceutically composition is administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by any combinations thereof.

62. The method of any one of claims 21-61, wherein the subject is a human.
